(19) United States
(12) Patent Application Publication
(10) Pub. No.: US 2007/0288014 A1
(43) Pub. Date: Dec. 13, 2007

(54) SPINE TREATMENT DEVICES AND METHODS

(76) Inventors: John H. Shadduck, Tiburon, CA (US); Csaba Truckai, Saratoga, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/758,596

(22) Filed: Jun. 5, 2007

Related U.S. Application Data
(60) Provisional application No. 60/811,093, filed on Jun. 6, 2006.

(51) Int. Cl. A61B 17/70

(52) U.S. Cl. 606/61; 606/90

(57) ABSTRACT

The invention relates generally to implant systems and methods for treating spine disorders, and more particularly to least invasive implant systems configured for re-distributing loads on a spine segment while still allowing spine flexion, extension, lateral bending and torsion. The implant system can include implants configured for spanning bi-lateral intercostal locations that can be introduced and implanted via posterior access to the spine through small bilateral incisions.

### CROSS-REFERENCE TO RELATED APPLICATIONS

This application claims the benefit of Provisional U.S. Patent Application No. 60/811,093 filed Jun. 6, 2006, the entire contents of which are incorporated herein by reference and should be considered a part of this specification.

### BACKGROUND OF THE INVENTION

1. Field of the Invention

The invention relates generally to implant devices, systems and methods for treating spine disorders, and more particularly relates to minimally invasive implant devices, systems and methods for re-distributing loads on a spine segment while still allowing spine flexion, extension, lateral bending and torsion.

2. Description of the Related Art

Thoracic and lumbar spinal disorders and discogenic pain are major socio-economic concerns in the United States affecting over 70% of the population at some point in life. Low back pain is the most common musculoskeletal complaint requiring medical attention; it is the fifth most common reason for all physician visits. The annual prevalence of low back pain ranges from 15% to 45% and is the most common activity-limiting disorder in persons under the age of 45.

Degenerative changes in the intervertebral disc often play a role in the etiology of low back pain. Many surgical and non-surgical treatments exist for patients with degenerative disc disease (DDD), but often the outcome and efficacy of these treatments are uncertain. In current practice, when a patient has intractable back pain, the physician's first approach is conservative treatment with the use of pain killing pharmacological agents, bed rest and limiting spinal segment motion. Only after an extended period of conservative treatment will the physician consider a surgical solution, which often is spinal fusion of the painful vertebral motion segment. Fusion procedures are highly invasive procedure that carries surgical risk as well as the risk of transition syndrome described above wherein adjacent levels will be at increased risk for facet and discogenic pain.

More than 150,000 lumbar and nearly 200,000 cervical spinal fusions are performed each year to treat common spinal conditions such as degenerative disc disease and spondylolisthesis, or misaligned vertebrae. Some 28 percent are multi-level, meaning that two or three vertebrae are fused. Such fusions “weld” unstable vertebrae together to eliminate pain caused by their movement. While there have been significant advances in spinal fusion devices and surgical techniques, the procedure does not always work reliably. In one survey, the average clinical success rate for pain reduction was about 75%; and long time intervals were required for healing and recuperation (3-24 months, average 15 months). Probably the most significant drawback of spinal fusion is termed the “transition syndrome” which describes the premature degeneration of discs at adjacent levels of the spine. This is certainly the most vexing problem facing relatively young patients when considering spinal fusion surgery.

Many spine experts consider the facet joints to be the most common source of spinal pain. Each vertebra possesses two sets of facet joints, one set for articulating to the vertebra above and one set for the articulation to the vertebra below. In association with the intervertebral discs, the facet joints allow for movement between the vertebrae of the spine. The facet joints are under a constant load from the weight of the body and are involved in guiding general motion and preventing extreme motions in the trunk. Repetitive or excessive trunkal motions, especially in rotation or extension, can irritate and injure facet joints or their encasing fibers. Also, abnormal spinal biomechanics and bad posture can significantly increase stresses and thus accelerate wear and tear on the facet joints.

Recently, technologies have been proposed or developed for disc replacement that may replace, in part, the role of spinal fusion. The principal advantage proposed by complete artificial discs is that vertebral motion segments will retain some degree of motion at the disc space that otherwise would be immobilized in more conventional spinal fusion techniques. Artificial facet joints are also being developed. Many of these technologies are in clinical trials. However, such disc replacement procedures are still highly invasive procedures, which require an anterior surgical approach through the abdomen.

Clinical stability in the spine can be defined as the ability of the spine under physiologic loads to limit patterns of displacement so as to not damage or irritate the spinal cord or nerve roots. In addition, such clinical stability will prevent incapacitating deformities or pain due to later spine structural changes. Any disruption of the components that stabilize a vertebral segment (e.g., disc, facets, ligaments) decreases the clinical stability of the spine.

Improved devices and methods are needed for treating dysfunctional intervertebral discs and facet joints to provide clinical stability, in particular: (i) implantable devices that can be introduced to offset vertebral loading to treat disc degenerative disease and facets through least invasive procedures; (ii) implants and systems that can restore disc height and foraminal spacing; and (iii) implants and systems that can re-distribute loads in spine flexion, extension, lateral bending and torsion.

### SUMMARY OF THE INVENTION

In accordance with one embodiment, an implant device for treating a spine segment including first and second vertebrae is provided. The implant comprises a body insertable in an intercostal space between adjacent vertebrae, the body comprising opposite end portions configured to engage adjacent transverse processes on the spine segment and an intermediate portion extending between said end portions, and at least one fixation portion extending from the body and configured to receive a fastener to fasten the body to the first and second vertebrae, wherein the implant body is configured to apply a distraction force on the transverse processes to thereby space apart the adjacent vertebrae.

In accordance with another embodiment, an implant device for treating a spine segment including first and second vertebrae is provided. The implant comprises an expandable body insertable in an intercostal space between adjacent vertebrae, the body comprising a medial portion positionable at least partially in the intercostal space between costal necks attached to the first and second vertebrae, and end portions on opposite ends of the medial portion, the end portions positionable on opposite sides of the costal necks from the medial portion, wherein the body is moveable from an unexpanded state configured to facilitate deployment of the implant in the intercostal space to an expanded state configured to off-load the spine segment.

In accordance with still another embodiment, a system for treating a spine segment including first and second vertebrae is provided. The system comprises a pair of implants configured for bi-lateral insertion in intercostal spaces between the costovertebral joints and costotransverse joints of the targeted spine segment to thereby off-load the spine segment.

In accordance with yet another embodiment, a method for treating a spine disorder is provided. The method comprises advancing an implant device through costotransversal foramens in two vertebrae so that a medial portion of the implant is disposed in an intercostal space between the costotransversal foramens of the vertebrae, and expanding the medial portion of the implant device to secure the implant device in the intercostal space.

In accordance with still another embodiment, a method for treating a spine segment including first and second vertebrae, the method comprising implanting at least one implant device configured to span an intercostal space between the costovertebral joint and costotransverse joint of the spine segment to thereby off-load the spine segment.

### BRIEF DESCRIPTION OF THE DRAWINGS

These and other features, aspects and advantages of the present inventions will now be described in connection with preferred embodiments, in reference to the accompanying drawings. The illustrated embodiments, however, are merely examples and are not intended to limit the inventions. The drawings include the following 33 figures, wherein:

FIG. 1 is a schematic posterior view of a spine segment with implants, in accordance with one embodiment;

FIG. 2 is a schematic view of the implants of FIG. 1 along the length of the patient's spine;

FIG. 3 is a schematic perspective view of a variation to the implant of FIG. 2, in accordance with another embodiment;

FIG. 4 is a perspective schematic view of another embodiment of an implant;

FIG. 5 is a schematic posterior view of a spine segment with the implants of FIG. 4 positioned in bi-lateral locations thereof;

FIG. 6 is a schematic side view of the spine segment of FIG. 5 with the implants of FIG. 4 in bi-lateral locations;

FIG. 7 is a schematic cross-sectional view of the implant of FIG. 4 along the length of the implant, in accordance with one embodiment;

FIG. 8 is a schematic cross-sectional view of an implant, in accordance with another embodiment;

FIGS. 9A-9B are schematic views of a patient's spine with another embodiment of an implant system deployed between adjacent transverse processes;

FIGS. 10A-10B are schematic perspective views of an implant of the system of FIGS. 9A-9B in non-expanded and expanded configurations; and

FIGS. 11A-11C are schematic views of one embodiment of a method of implanting the system of FIGS. 9A-9B in a minimally invasive procedure.

### DETAILED DESCRIPTION OF THE PREFERRED EMBODIMENT

Embodiments disclosed herein provide a minimally invasive surgery (MIS) implant system for off-loading a spine segment (e.g., first and second adjacent vertebrae) by placing spacer-like implant devices between vertebrae of the spine segment (e.g., in intercostals spaces 105 between first 108 and second 108′ adjacent vertebrae). Intercostal spaces, as used herein, mean spaces between vertebrae 108, 108′ outward from the costovertebral joints between ribs 106, 106′ and the corresponding vertebrae 108, 108′, and includes spaces between the transverse processes 122, 122′, spaces between costal heads 121, and spaces between costal necks 124, of the spine segment. The system is a non-fusion type of system, to thereby provide dynamic stabilization of a vertebra 108 in a targeted spine segment, while at the same time off-loading forces on the disc and facets. Advantageously, the implant system also can be used for treating scoliosis.

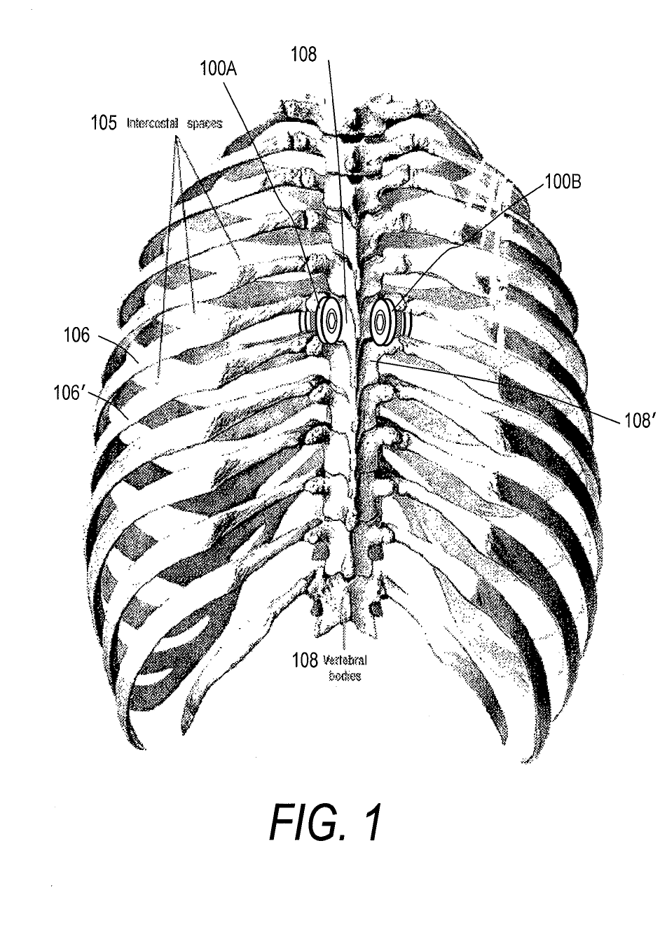

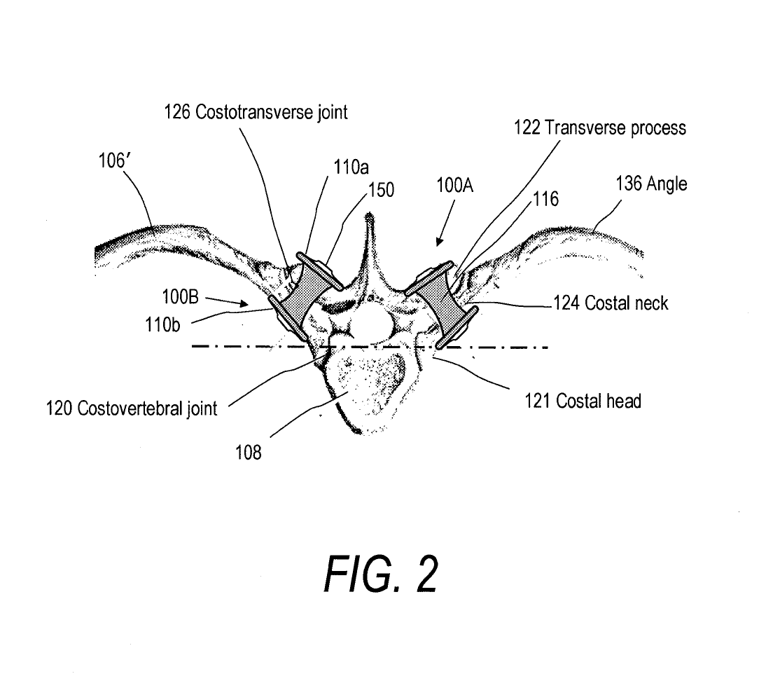

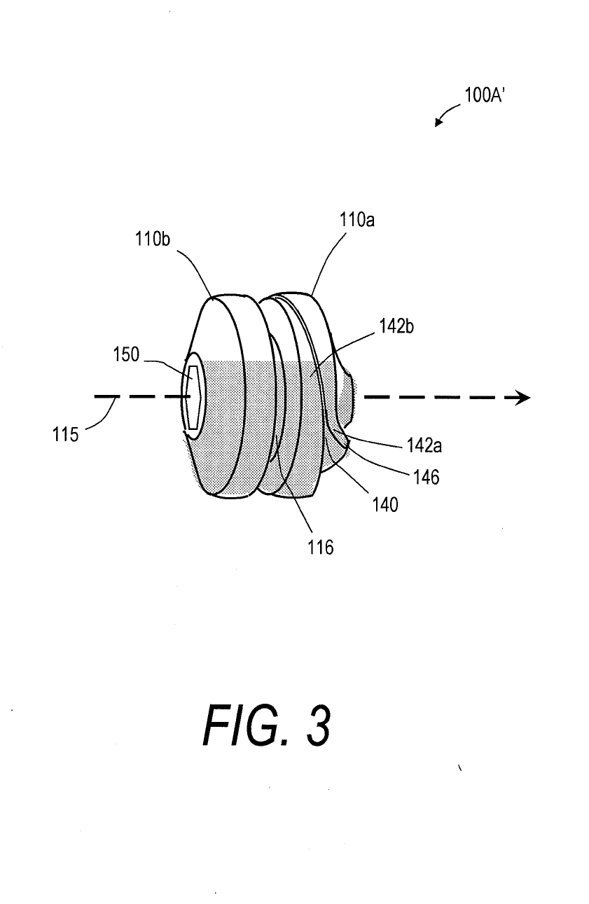

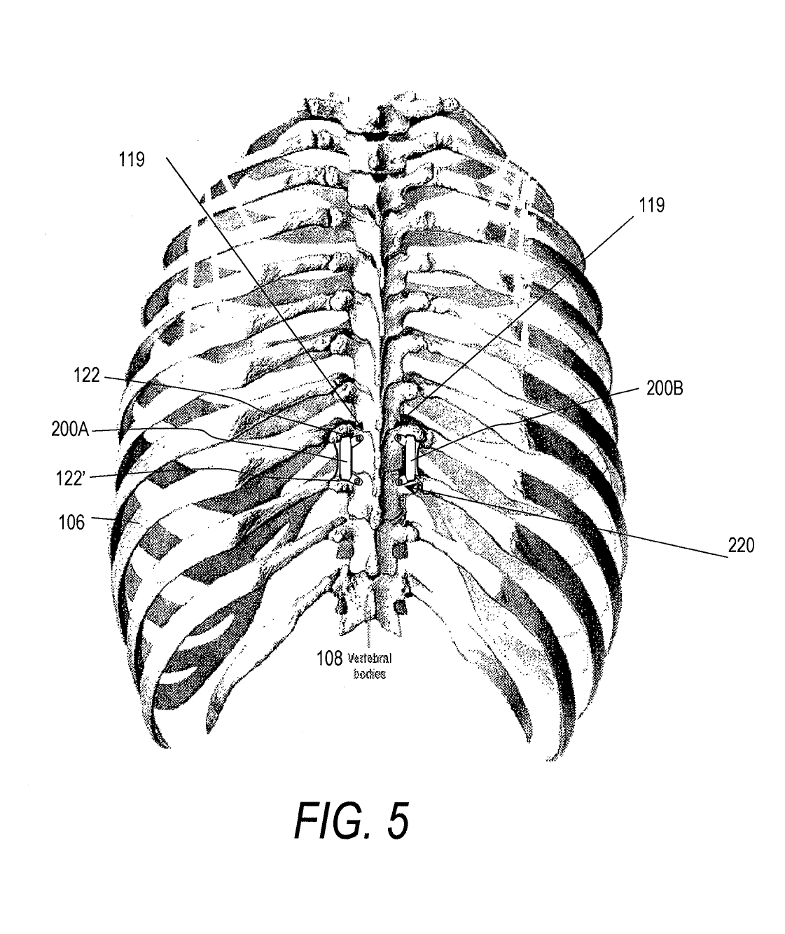

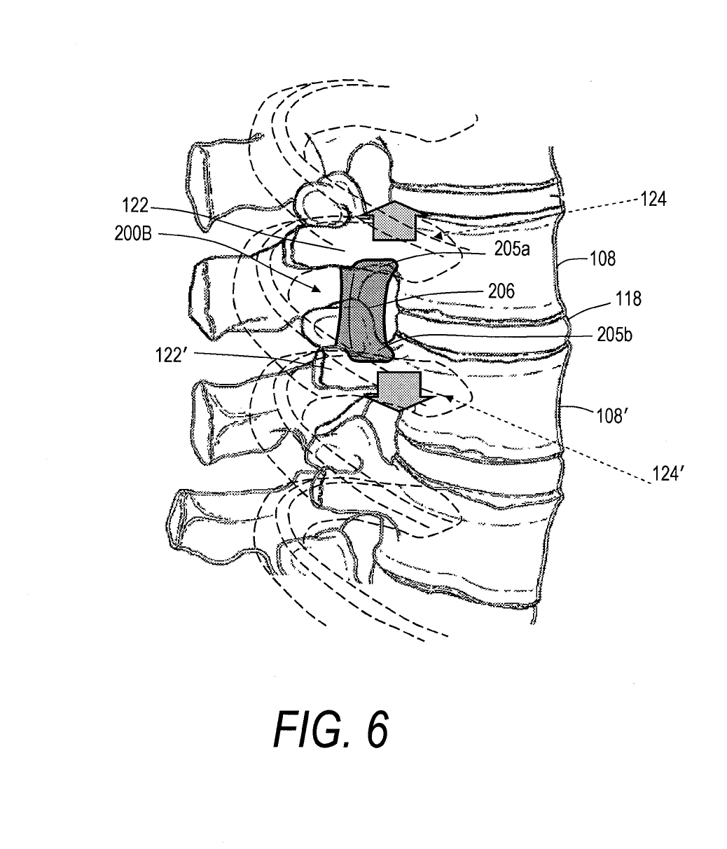

FIGS. 1 and 2 illustrate one embodiment of a bi-lateral paired implant system with implant bodies or devices 100A and 100B. The implants 100A, 100B can be introduced in a minimally invasive posterior approach through small bilateral incision(s) in a patient's back. In one embodiment, the devices 100A and 100B have a generally “H”-shaped cross-section in a repose state. However, the implants 100A, 100B can have other suitable cross-sections. In the illustrated embodiment, the devices or implants 100A and 100B each include first and second flange ends or collar portions 110a and 110b relative to a longitudinal insertion axis indicated at 115 (see FIG. 3). Each implant 100A, 100B also has a medial portion 116 intermediate the end portions 110a and 110b that has a reduced cross-section or saddle for controlling the dimension of a targeted intercostal space 105 between superior rib 106 and inferior rib 106′ (e.g., the medial portion 116 has a smaller transverse cross-section than the flange ends 110a, 110b). The medial or saddle portion 116 can have a predetermined cross-sectional dimension transverse to the axis of the implant 100A, 100B for engaging and spacing apart selected bone portion processes to reduce loads on the disc 118 and facet joints 119 (see FIGS. 5 and 6) to increase vertebral spacing (e.g., the medial portion 116 can have a cross-sectional dimension that spaces apart vertebrae 108, 108′ by a desired amount), which can thereby alleviate compression of nerves. In one embodiment, the flange ends 110a, 110b and the medial portion 116 form a unitary body. In another embodiment, the implants can be modular with separate flange ends and medial portions.

The implant system includes paired devices 100A and 100B that can span intercostal spaces 105 in bi-lateral locations outwardly, relative to the spine, from the costovertebral joints 120. For example, the locations for implantation of the devices can be between the transverse processes 122 and the costal necks 124, and between the costotransverse joints 126 and the costotransverse joint 126, as indicated in FIG. 2. The devices also can be implanted between the costotransversal foramens 135, or between the transverse processes 122 without substantial costal engagement. Still more generally, the devices can be implanted in an intercostal space inwardly from the costal angles 136.

In the illustrated embodiment, the implant bodies are adapted to engage both the transverse processes 122 and costal necks 124. By engaging the transverse processes 122, the various ligaments are preserved for maintaining spine stability. Additionally, since there is no need to remove bone material, the patient's extension and flexion capabilities are preserved, and lateral bending and axial rotation remain substantially the same. The system advantageously increases disc height and foraminal spacing between vertebrae to alleviate pain.

In another embodiment, as shown in FIG. 3, an implant 100A′ is adapted for minimally invasive helical insertion, for example in the thoracic spine. The implant 100A′ is similar to the implants 100A, 100B discussed above. Thus, the reference numerals used to designate corresponding components in the implant 100A′ and the implant 100A are identical.

Of particular interest, referring to FIG. 3, the implant 100A′ has a first body portion or flange 110a end of a resilient material, such as a high modulus rubber with a helical slot or discontinuity 140 therein that extends from the body periphery inwardly to provide body portions 142a and 142b on either side of helical discontinuity 140 about axis 115. However, other suitable resilient materials can be used. The body portion 110a has a lip 146 for allowing helical engagement of the implant 100A′ with a vertebral portion (e.g., a transverse process) upon insertion of the implant 100A′. The helical discontinuity 140 can extend inwardly to the central shaft portion or saddle 116 of the implant. In one embodiment, the implant 100A′ can be helically advanced relative to axis 115 and inserted in between adjacent vertebrae 108, 108′ (see e.g., FIG. 1), wherein the cross-section of the medial or saddle region 116 provides a spacer to maintain an intercostal space (e.g., between transverse processes and costal necks of the vertebrae).

The device or implant 100A, 100A′ can have a form 150 (FIGS. 2, 3) such as, for example, a hex form for cooperating with a helical driving instrument (not shown) used to deploy the implant 100A, 100A′. In other embodiments, the form can be a threaded, polygonal or slotted form suitable for engaging a driving instrument. The transverse cross-section of the medial body or saddle region 116 of implant device 100A, 100A′ that can function as a spacer can have any suitable shape, such as, for example, round, rectangular or oval. In one embodiment, the medial region 116 can have a core portion of a metal or hard polymer and a surface layer of a slightly compressible and resilient material adapted to engage (e.g., grip) the bone surfaces (e.g., transverse processes 122, 122′).

The above embodiments include implant devices that have unitary bodies. However, in other embodiments, the implant devices can have multiple part bodies that can be assembled in situ to provide the configuration shown, for example, in FIGS. 1-3, as can be understood from the art. For example, an implant can be assembled from a central shaft portion and first and second flange end portions. Additionally, the implant devices discussed herein, such as the helically-driven implant of FIG. 3 also can be configured for minimally invasive implantation between spinous processes through a single incision.

Thus, one embodiment of a method for reducing physiologic loads on facet joints includes providing an axially-extending implant body with first and second spaced apart flange portions and an intermediate saddle or shaft portion wherein the first flange portion is of a resilient material having a helical discontinuity therein; and helically advancing the body between adjacent bone portions (e.g., transverse processes, costal necks, etc.), wherein the helical discontinuity allows the first flange portion to be screwed through the intercostal space. Further, the method can include implanting the body through a single small incision overlying the intercostal space. The method can also include advancing the body over a guide member. Further, the method can also include adjusting the height of the intercostal spacer portion in situ at the time of surgery or at a later date.

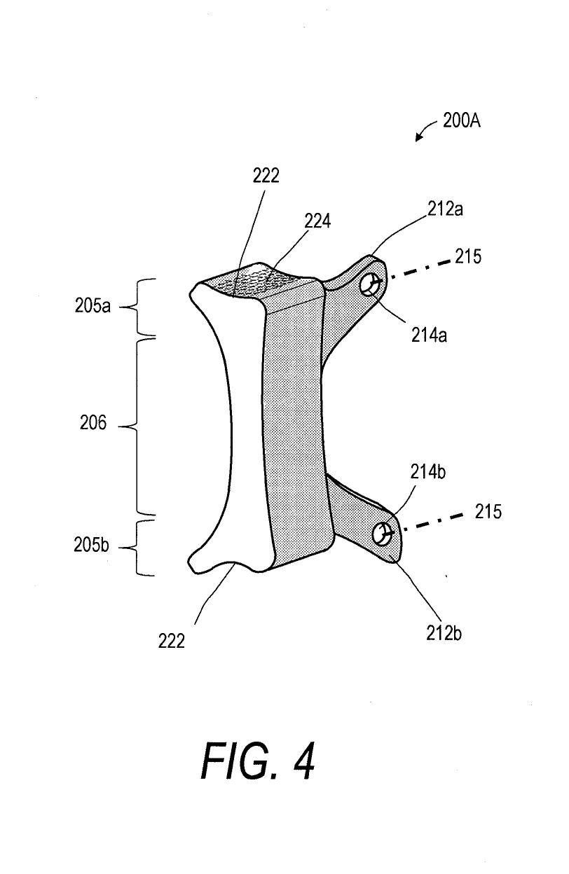

FIGS. 4-6 illustrate another embodiment of an implant system with implant bodies or devices 200A, 200B. In the illustrated embodiment, the implant bodies 200A, 200B off-load the disc 118 and facet joints 119 in any lumbar, thoracic or cervical region of the spine by providing a spacer that engages adjacent transverse processes 122 and 122′ and optionally costal necks 124 and 124′ (see FIG. 6). One embodiment of the implant body 200A is shown in FIG. 4, wherein the body 200A has superior and inferior end portions 205a and 205b for engaging the transverse processes 122 and 122′. The implant body 200A has an intermediate body portion 206 extending between the end portions 205a and 205b. The implant body 200A includes first and second (e.g., superior and inferior) fixation or projecting portions 212a and 212b, each having an opening 214a, b therein aligned with an axis 215 thereof for receiving a bone screw 220 (see FIG. 5) or other type of transpedicular member that is adapted to be fixed into a pedicular bore or parapedicular bore. It should be appreciated that the fixation portions (212a and 212b) can either extend from the device body 200A at any suitable angle or be substantially integral to the device body, and can be flexible or rigid as adapted for the particular targeted space between transverse processes 122 and 122′.

In one embodiment, as depicted in FIGS. 4 and 6, implant bodies 200A and 200B can have a concavity or saddle portion 222 in each of the superior and inferior end portions 205a and 205b for engaging transverse processes 122 and 122′. Additionally, in one embodiment, the surface of the concavity can also have a texture 224 for engaging the bone surface.

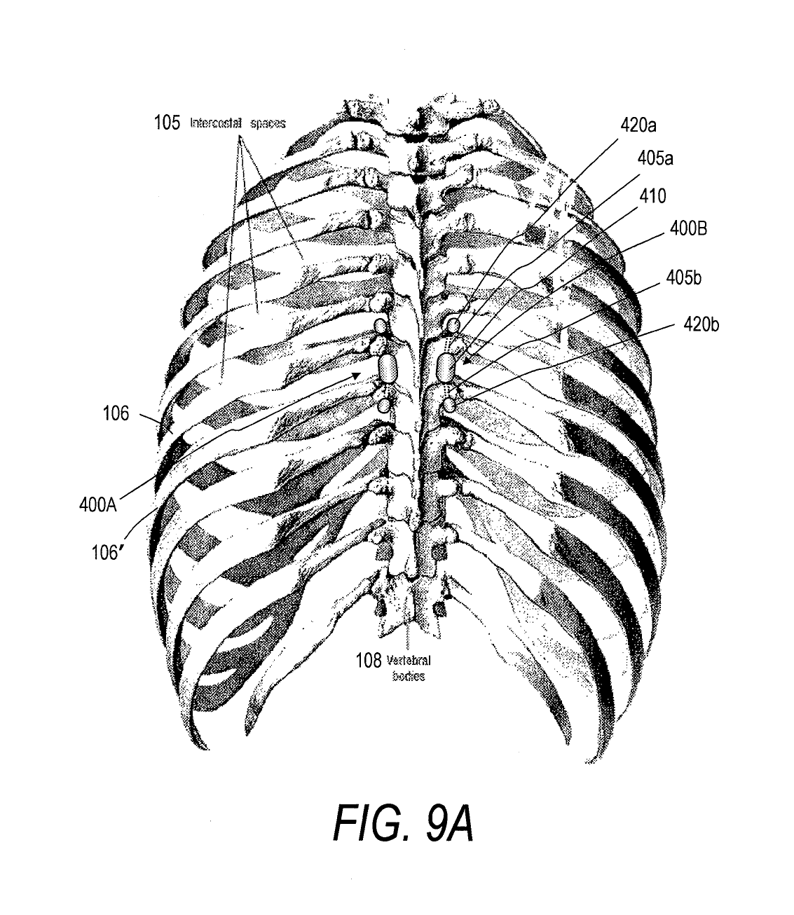

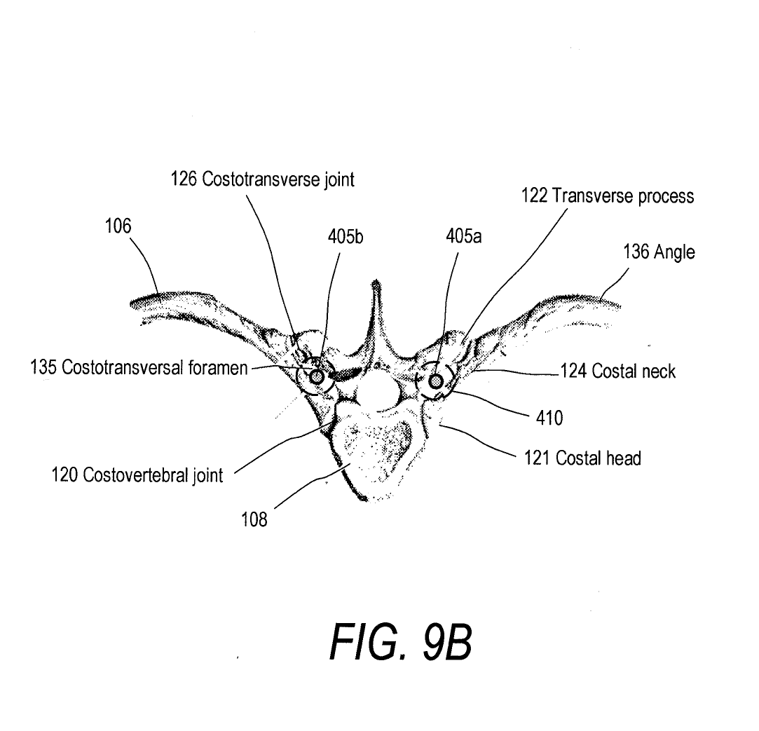

In the embodiments of FIGS. 4-6, the implant bodies 200A and 200B are maintained between the engaged transverse processes 122 and 122′ at least in part by the concavity 222 and by the fixation portions that have a bone screw or other bone-penetrating member therein. In other embodiments, of the implant devices can have additional or alternative fixation mechanisms, such as a tether element (such as tether 405a, below) that can extend through ligaments and the costotransverse foramen 135 (see FIG. 9B) or a strap (not shown) that can extend around the transverse process 122 and costal neck 124 (see FIG. 9B).

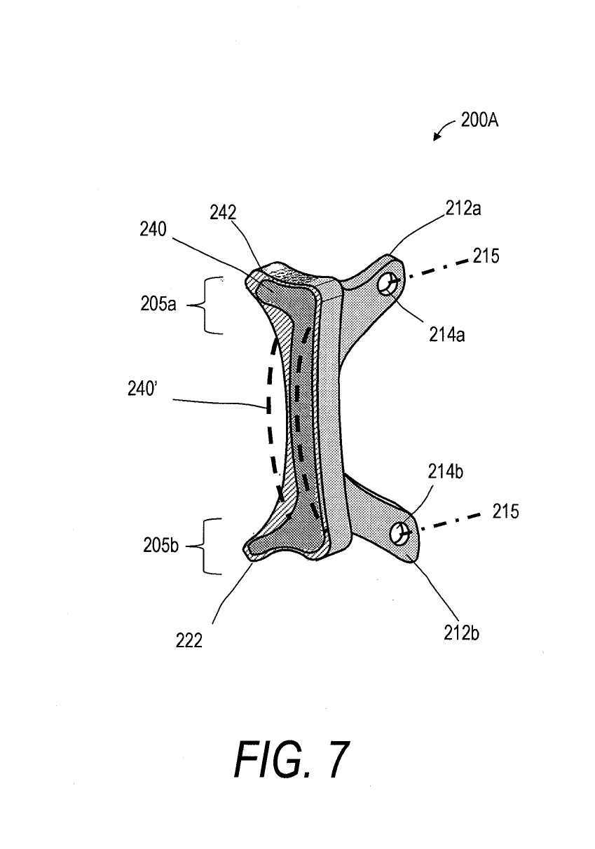

FIG. 7 illustrates a cross-sectional view of the implant body 200A along the length of the implant 200A that shows a metal core portion 240 with a polymeric portion 242 about the metal core 240. The metal core 240 can include a spring element that absorbs loads by deflecting from a rest position to a flexed position 240′, as indicated in phantom in FIG. 7. The polymeric portion 242 can also be of a resilient material that absorbs loads. Any suitable resilient material can be used.

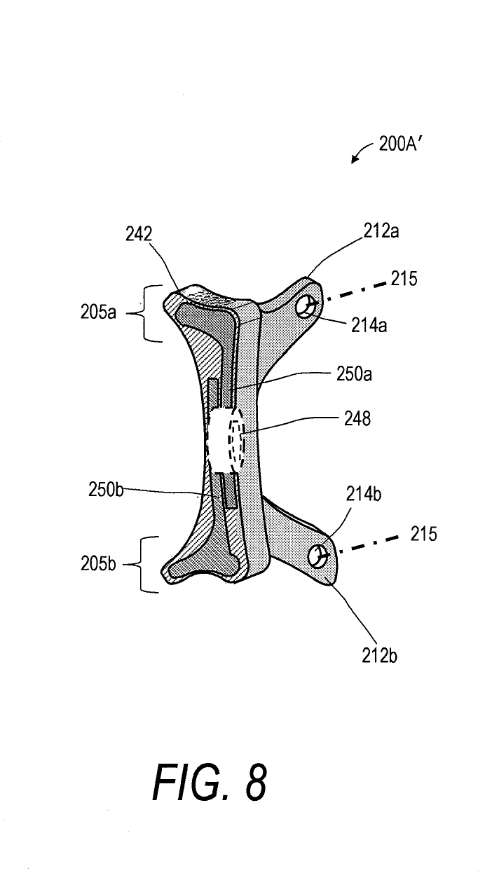

FIG. 8 illustrates a cross-sectional view of another embodiment of an implant body 200A′ along the length of the implant 200A′ that is similar to that of FIGS. 4-6 except that the medial portion 206 includes length-adjustment mechanism. In the illustrated embodiment, the length-adjustment mechanism includes a rotatable screw 248 that secures first and second metal core portions 250a and 250b relative to one another along the length of the implant 200A′ to increase or decrease the height of the implant 200A′. In one embodiment, the screw 248 can operate as a gear to move the first and second metal core portions 250a and 250b relative to each other. In another embodiment, the screw can clamp the core portions 250a and 250b together after being adjusted manually within an elastomeric polymer coating 242. However, other suitable length-adjustment mechanisms known in the art can be used, including mechanical linkages, jacks, screws, gears, toggles, cams, pin-type hinges, living hinges, mechanically deformable metals and polymers, fluid-expandable metal bellows, expandable distensible structures such as balloons, bladders, bellows and the like, osmotic materials that expand upon fluid absorption such as suitable polymers, seaweed and the like, and shape memory metals and polymers.

In another embodiment (not shown) similar to that of FIGS. 4-8, the implant body 200A can include an interior chamber defined by an at least partly expandable surface. The interior chamber can have a fitting or connector allowing the implant to couple to a flowable polymer inflow source. The interior chamber can also have a thermal emitter to heat and cure the polymer flowed into the implant chamber. Additionally, the implant body 200A can have a connector for coupling the implant to an energy source to provide energy to the thermal emitter to cure and harden the inflow polymer on demand.

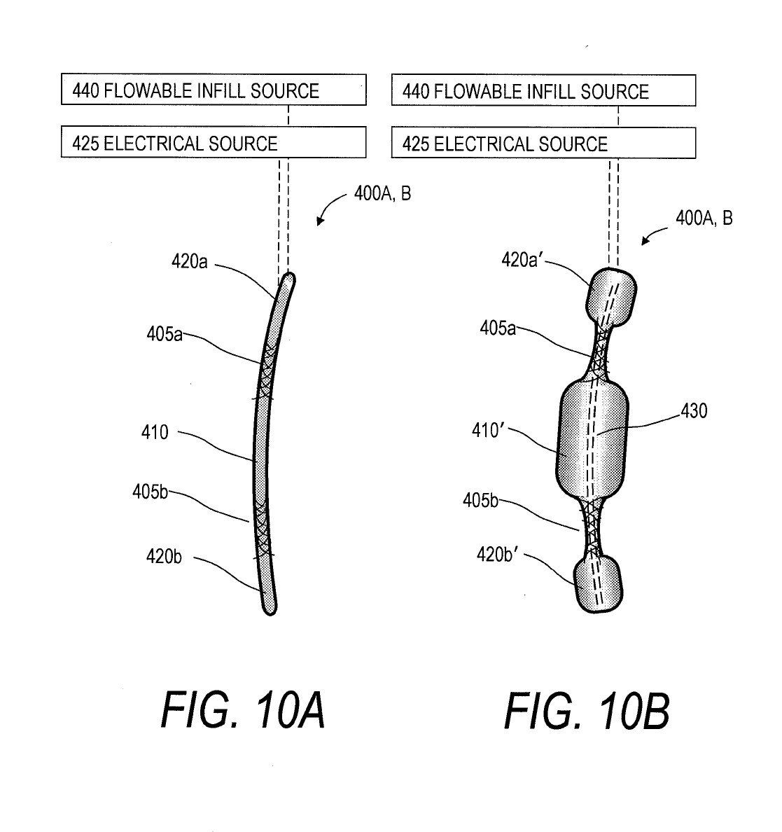

FIGS. 9A-9B and 10A-10B illustrate another embodiment of a dynamic stabilization implant system for off-loading discs and facet joints, wherein implant bodies 400A and 400B are introduced in bi-lateral locations between transverse processes 122 and costal necks 124 and secured in place by tether portions 405a and 405b that extend through the ligaments and costotransversal foramens 135. In particular, each implant body 400A and 400B has a medial body portion 410 of a substantially rigid material to act as a spacer in the intercostal space. The medial body portion 410 can have a fixed vertical dimension with any suitable end configuration (e.g., flat, concave, convex, textured, abrasive, with projections and the like) for engaging the bone-ligament surface. In one embodiment, the medial body portion 410 includes therein a flexible metal spring-like element that deform deforming under loads, as described above in connection with the implant 200A. In another embodiment, the medial body portion 410 includes a flexible metal core and resilient polymeric coating. In another embodiment, the medial body portion 410 includes an interior chamber for fluid expansion of the body portion 410 to engage and distract the intercostal space. Further, the medial body portion 410 can also have a heating element, a connector for coupling to an energy source and a connector for coupling to a source of hardenable inflow material, as described above in other implant embodiments.

The implant bodies 400A and 400B of FIGS. 9A-10B further include first and second end portions 420a and 420b coupled to tether portions 405a and 405b wherein each of the first and second end portions 420a and 420b are expandable and have an interior chamber for fluid expansion to secure the implants 400A and 400B in the intercostal spaces.

As shown in FIGS. 10A, 10B, the implant bodies 400A, 400B can be expanded from an unexpanded configuration (see FIG. 10A) to an expanded configuration (see FIG. 10B). In the unexpanded configuration, the end portions 420a, 420b, tether portions 405a, 405b and body portion 410 have generally the same configuration and the implant bodies 400A, 400B have a rod-like or linear configuration that advantageously allows for simplified deployment of the implant bodies 400A, 400B through, for example, the costotransversal foramen 135 (as shown in FIG. 9B). In the expanded configuration, the tether portions 405a, 405b retain generally the same cross-section as in the unexpanded configuration. However, the end portions 420a′, 420b′ and body portion 410′ have larger cross-sections than in the unexpanded configuration. The implant bodies 400A, 400B can be expanded by delivering an infill material (e.g., polymer, resin, etc.) from a flowable infill source 440 into the implant body 400A, 400B. The system can also include an electrical source 425 coupled to the implant body 400A, 400B for delivering thermal energy to the infill material (e.g., via heating elements 430 in the implant body) to harden the material within the implant body 400A, 400B.

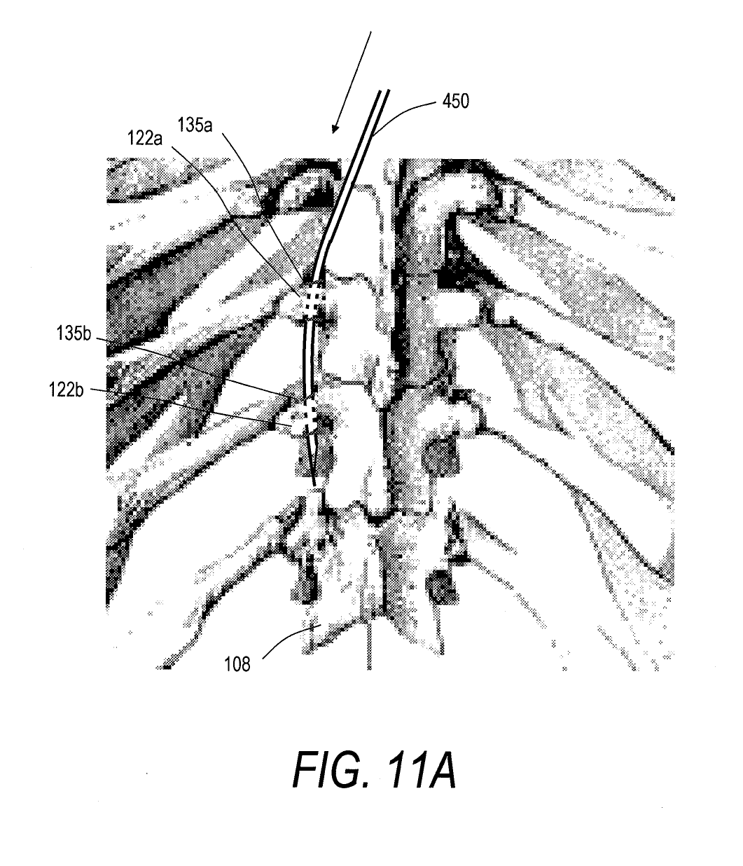

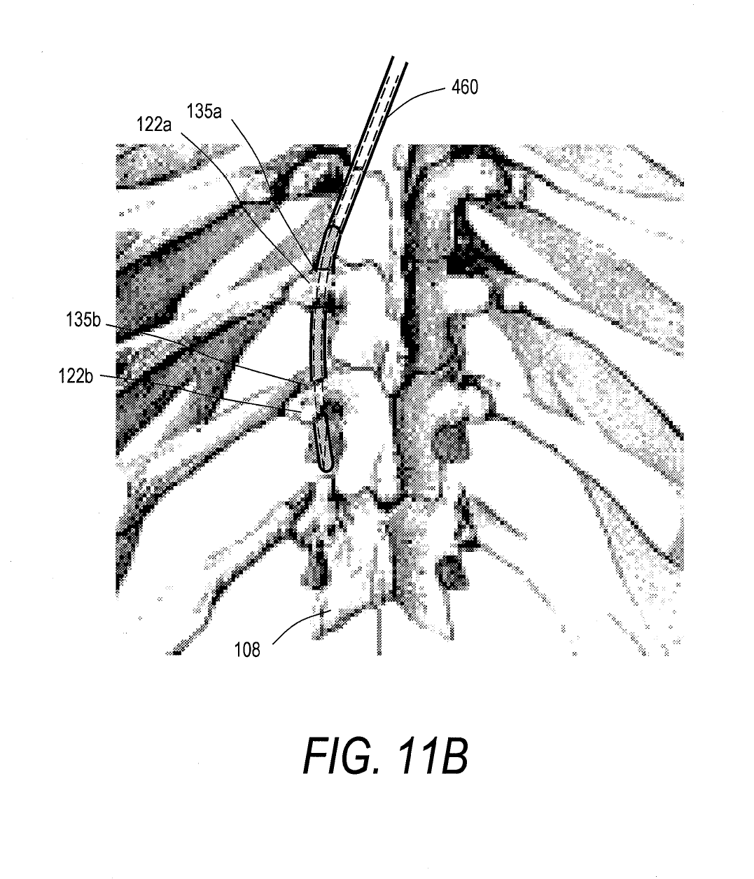

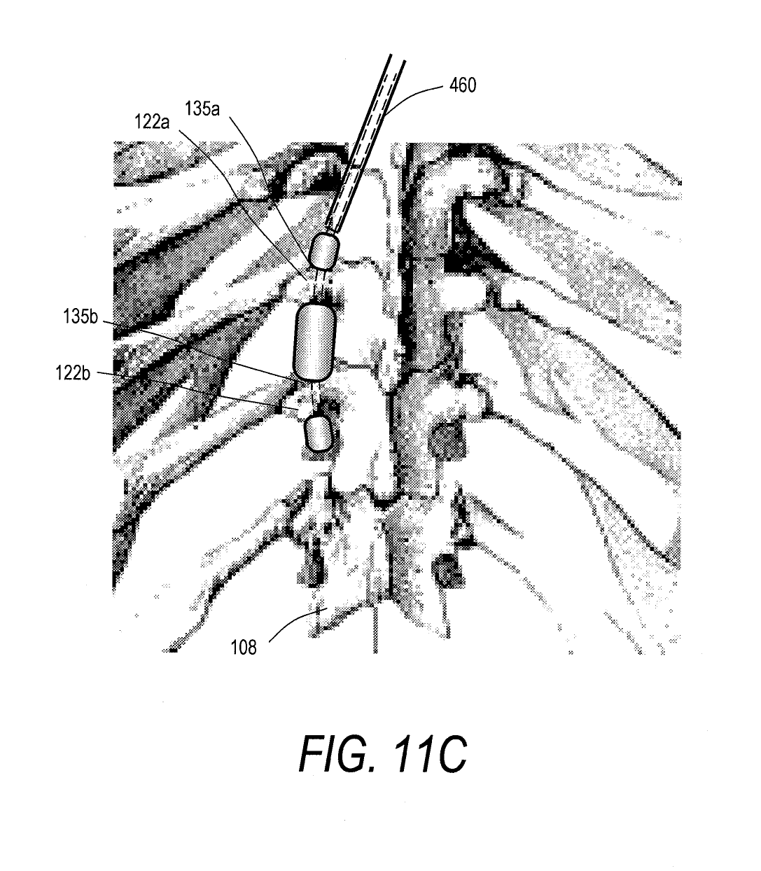

FIGS. 11A-11C illustrate a method for implanting the device bodies 400A and 400B of FIGS. 9A-10B. It can be seen in FIG. 11A that a radiopaque guide member 450 is introduced through at least two adjacent costotransversal foramens 135. Thereafter, in FIG. 11B, the device body 400A is introduced in a non-expanded configuration via a cannula 460. In FIG. 11C, the device is moved to the deployed configuration is which first the medial portion 410 is expanded, in accordance with one embodiment. Thereafter, the first and second end portions 420a and 420b coupled to tether portions 405a and 405b are expanded to secure the implant in place. The inflow material preferably is a polymer that can be hardened to a controlled modulus that has a selected resilience to allow the implant to act akin to a “shock absorber” in spine flexion and extension. Advantageously spine rotation will still be allowed after the bi-lateral implants 400A, 400B are deployed.

Certain embodiments described above provide new ranges of minimally invasive, reversible treatments that form a new category between traditional conservative therapies and the more invasive surgeries, such as fusion procedures or disc replacement procedures. One embodiment includes an implant system configured for spanning bi-lateral intercostal locations that can be introduced and implanted via posterior access in a patient's back formed by small bilateral incisions.

Certain embodiments include implant systems that can be implanted in a very minimally invasive procedure, and require only small bilateral incisions in a posterior approach. A posterior approach is highly advantageous for patient recovery. In some embodiment, the implant systems are “modular” in that separate implant components are used that can be implanted in a single surgery or in sequential surgical interventions. Certain embodiments of the inventive procedures are for the first time reversible, unlike fusion and disc replacement procedures. Additionally, embodiments of the invention include implant systems that can be partly or entirely removable. Further, in one embodiment, the system allows for in-situ adjustment requiring, for example, a needle-like penetration to access the implant.

In certain embodiments, the implant system can be considered for use far in advance of more invasive fusion or disc replacement procedures. In certain embodiments, the inventive system allows for dynamic stabilization of a spine segment in a manner that is comparable to complete disc replacement. Embodiments of the implant system are configured to improve on disc replacement in that it can augment vertebral spacing (e.g., disc height) and foraminal spacing at the same time as controllably reducing loads on facet joints—which complete disc replacement may not address. Certain embodiments of the implant systems are based on principles of a native spine segment by creating stability with a tripod load receiving arrangement. The implant arrangement thus supplements the spine's natural tripod load-bearing system (e.g., disc and two facet joints) and can re-distribute loads with the spine segment in spine torsion, extension, lateral bending and flexion.

Of particular interest, since the embodiments of implant systems are far less invasive than artificial discs and the like, the systems likely will allow for a rapid regulatory approval path when compared to the more invasive artificial disc procedures.

Other implant systems and methods within the spirit and scope of the invention can be used to increase intervertebral spacing, increase the volume of the spinal canal and off-load the facet joints to thereby reduce compression on nerves and vessels to alleviate pain associated therewith.

Although these inventions have been disclosed in the context of a certain preferred embodiments and examples, it will be understood by those skilled in the art that the present inventions extend beyond the specifically disclosed embodiments to other alternative embodiments and/or uses of the inventions and obvious modifications and equivalents thereof. In addition, while a number of variations of the inventions have been shown and described in detail, other modifications, which are within the scope of the inventions, will be readily apparent to those of skill in the art based upon this disclosure. It is also contemplated that various combinations or subcombinations of the specific features and aspects of the embodiments may be made and still fall within one or more of the inventions. Accordingly, it should be understood that various features and aspects of the disclosed embodiments can be combine with or substituted for one another in order to form varying modes of the disclosed inventions. Thus, it is intended that the scope of the present inventions herein disclosed should not be limited by the particular disclosed embodiments described above.

What is claimed is:

1. An implant device for treating a spine segment including first and second vertebrae, the implant comprising:
a body insertable in an intercostal space between adjacent vertebrae, the body comprising opposite end portions configured to engage adjacent transverse processes on the spine segment and an intermediate portion extending between said end portions; and
at least one fixation portion extending from the body and configured to receive a fastener to fasten the body to the first and second vertebrae,
wherein the implant body is configured to apply a distraction force on the transverse processes to thereby space apart the adjacent vertebrae.

2. The implant device of claim 1, wherein each of the end portions comprises a concave portion configured to engage the transverse processes.

3. The implant device of claim 1, wherein each of the end portions comprises a textured surface configured to engage the transverse processes.

4. The implant device of claim 1, wherein the body comprises at least one metal core portion disposed within at least one polymeric portion.

5. The implant device of claim 4, wherein the at least one metal core portion comprises a spring configured to deflect to absorb load forces applied to the body.

6. The implant device of claim 1, further comprising a length-adjustment mechanism disposed in the body and configured to adjust the length of the body.

7. The implant device of claim 6, wherein the length-adjustment mechanism comprises a first and a second core metal portions moveable relative to each other to adjust a length of the body, the core metal portions fastenable to each other with a fastener to substantially maintain a selected length.

8. An implant device for treating a spine segment including first and second vertebrae, the implant comprising:
an expandable body insertable in an intercostal space between adjacent vertebrae, the body comprising a medial portion positionable at least partially in the intercostal space between costal necks attached to the first and second vertebrae, and end portions on opposite ends of the medial portion, the end portions positionable on opposite sides of the costal necks from the medial portion,
wherein the body is moveable from an unexpanded state configured to facilitate deployment of the implant in the intercostal space to an expanded state configured to off-load the spine segment.

9. The implant device of claim 8, further comprising tether portions that couple the end portions to the medial portion.

10. The implant device of claim 8, wherein at least one of the medial portion and end portions defines a chamber configured to receive a fluid to expand the body.

11. The implant device of claim 10, wherein the fluid is a hardenable material.

12. The implant device of claim 8, further comprising a heating element disposed in the body, the heating element removably coupleable to an energy source configured to deliver energy to an infill material deliverable into the body from a flowable infill source removably coupleable to the body to harden the infill material.

13. A system for treating a spine segment including first and second vertebrae, the system comprising:
a pair of implants configured for bi-lateral insertion in intercostal spaces between the costovertebral joints and costotransverse joints of the targeted spine segment to thereby off-load the spine segment.

14. The system of claim 13, wherein each of the implants comprises an intermediate portion positionable in the intercostal space, and a pair of end portions on opposite sides of the intermediate portion, the end portions positionable on opposite sides of the costovertebral joints from the intermediate portion, the medial portion configured to engage the vertebrae.

15. The system of claim 14, wherein at least one of the implants includes a helical configuration configured to allow for helical insertion of the implant into the intercostal space.

16. The system of claim 14, wherein at least one of the end portions and intermediate portion of the implant are expandable from an unexpanded state configured to facilitate insertion of the implant into the intercostal space to an expanded configuration configured to engage the vertebrae to thereby off-load the spine segment.

17. The system of claim 13, wherein at least one of the implants is mechanically expandable.

18. The system of claim 13, wherein at least one of the implants is expandable via introduction of a fluid therein.

19. The system of claim 18, wherein the fluid comprises a hardenable material.

20. The system of claim 19, wherein the fluid comprises a curable polymer.

21. The system of claim 18, further comprising an energy source removably coupleable to the implant to deliver energy to the hardenable material to harden the material.

22. The system of claim 13, wherein at least one of the implants comprises a substantially rigid intercostal portion.

23. The system of claim 13, wherein at least one of the implants comprises a substantially resilient intercostal portion.

24. The system of claim 13, wherein at least one the implants has a unitary body.

25. A method for treating a spine disorder, comprising:
advancing an implant device through costotransversal foramens of two vertebrae so that a medial portion of the implant is disposed in an intercostal space between the costotransversal foramens of the vertebrae; and
expanding the medial portion of the implant device to secure the implant device in the intercostal space.

26. The method of claim 25, wherein advancing the implant includes inserting the implant via a minimally invasive posterior approach through a small incision in a patient's back.

27. The method of claim 25, wherein advancing the implant device includes positioning end portions of the implant device on opposite sides of the costotransversal foramens from the medial portion such that tether portions connecting the end portions to the medial portion extend through the costotransversal foramens of the vertebrae.

28. The method of claim 27, further comprising expanding the end portions of the implant.

29. The method of claim 25, wherein expanding includes delivering a flowable material into the implant.

30. The method of claim 29, further comprising delivering energy to the flowable material in the implant to harden said material.

31. A method for treating a spine segment including first and second vertebrae, the method comprising implanting at least one implant device configured to span an intercostal space between the costovertebral joint and the costotransverse joint of the spine segment to thereby off-load the spine segment.

32. The method of claim 31, wherein implanting the at least one implant device includes implanting first and second implant devices in intercostal spaces between the costovertebral joint and the costotransverse joint of the spine segment.

33. The method of claim 32, wherein implanting the first and second implant devices comprises implanting the devices bi-laterally on opposite sides of the spine segment.
